(19) United States
(12) Patent Application Publication
(10) Pub. No.: US 2013/0274837 A1
(43) Pub. Date: Oct. 17, 2013

(54) Systems and Methods to Enhance Optical Transparency of Biological Tissues for Photobiomodulation

(71) Applicant: Babak Nemati, La Jolla, CA (US)

(72) Inventor: Babak Nemati, La Jolla, CA (US)

(21) Appl. No.: 13/914,813

(22) Filed: Jun. 11, 2013

Related U.S. Application Data
(63) Continuation-in-part of application No. 13/352,246, filed on Jan. 17, 2012.
Application No. 13/352,246 is a continuation of application No. 11/262,082, filed on Oct. 27, 2005, now Pat. No. 8,096,982.
Application No. 11/262,082 is a continuation-in-part of application No. 09/777,640, filed on Feb. 7, 2001, now abandoned.
Application No. 09/777,640 is a division of application No. 09/177,340, filed on Oct. 23, 1998, now Pat. No. 6,018,392.
(60) Provisional application No. 61/689,777, filed on Jun. 13, 2012.

(51) Int. Cl. A61N 5/06

(52) U.S. Cl. CPC A61N 5/0613
USPC 607/88

(57) ABSTRACT

Systems and methods for photobiomodulation of biological processes using invasive or non-invasive chemical clarification of biological tissues are provided to improve optical transmission of light energy in sub-epidermal tissue. The chemical clarification of in vivo biological tissues provides at least partial optical clarification of such tissues by applying a clarifying agent to the sub-epidermal tissue to temporarily replace water and other fluids from such tissues. Light energy is then applied to the clarified tissues, providing for deeper penetration of the light energy and more effective photobiomodulation. Lower power and wavelengths of light can be administered at high fluences into the tissue, at greater depths, and induce biological effects that are more pronounced than previously observed.

### RELATED APPLICATIONS INFORMATION

This application claims priority from U.S. Provisional Application No. 61/689,777 filed on Jun. 13, 2012, and is also a continuation-in-part of U.S. patent application Ser. No. 13/352,246, filed Jan. 17, 2012, now pending, which is a continuation of U.S. patent application Ser. No. 11/262,082, filed Oct. 27, 2005, now U.S. Pat. No. 8,096,982, which is a continuation-in-part of U.S. patent application Ser. No. 09/777,640, filed Feb. 7, 2001, now abandoned, which is a divisional of U.S. patent application Ser. No. 09/177,348, filed on Oct. 23, 1998, now U.S. Pat. No. 6,219,575, all of which are incorporated herein by reference in their entirety as if set forth in full.

### FIELD OF THE INVENTION

The field of the invention relates to systems and methods for photobiomodulation of biological processes using invasive or non-invasive application of chemicals to biological tissues to improve optical transmission of light energy in sub-epidermal tissue.

### BACKGROUND OF THE INVENTION

Photobiomodulation generally refers to the application of light onto biological tissue to cause a variety of therapeutic effects. The advent of new light sources, such as light emitting diodes (LEDs) and low energy lasers has brought renewed interest to light therapy. In photobiomodulation, low level light sources are applied to biological tissues at particular wavelengths, leading to biological effects that are not caused by thermal or evaporative (ablative) tissue interaction with light. The effect is primarily stimulatory, although the exact mechanisms of low level light interaction with the tissue are not fully understood. Data suggests that empirically low level light has produced objective alteration in the skin, including increased collagen production, increased fibroblast production and increased macrophage activity, amongst others. Optical properties of biological tissues suggest that most key components of biological tissue serve as chromophores that are more responsive to lower wavelength light, but such light may not penetrate deep enough into the tissue to produce the desired results.

Chemical agents may be delivered to a target biological tissue in order to increase the optical transmission through this tissue on a transient basis. While the precise mechanism of increasing optical transmission through biological tissues is not well understood, it is believed that the administered chemical agent displaces the aqueous interstitial fluid of the tissue, thereby effectively altering the interstitial refractive index, of the tissue. If the index of refraction of the administered chemical is closer to that of the other components of the tissue, the introduction of this chemical will result in a reduction in the heterogeneity of the refractive indices of the tissue, which in turn reduces the level of scattering within the tissue. Since optical attenuation through the tissue is primarily due to absorption and scattering, a substantial change in scattering dramatically affects the optical attenuation, and consequently optical transmission, characteristics of most biological tissues.

### SUMMARY OF THE INVENTION

Embodiments described herein are directed to systems and methods for photobiomodulation of biological processes using invasive or non-invasive chemical modification of biological tissues to improve optical transmission of low level light energy in sub-surface tissue. The chemical clarification of in vivo biological tissues provides at least partial optical clarification of such tissues by applying a clarifying agent to the interstitial space to temporarily replace water and other fluids from such tissues. Low level light therapy (LLLT) is then performed on the clarified tissues, providing for deeper penetration of the light energy and more effective photobiomodulation. Most of the key chromophores of tissues absorb light in lower wavelengths and as such it is reasonable to expect that LLLT effects would be more pronounced, if there is an ability to deliver lower wavelength light to the bulk of the tissue at higher doses and greater depths, thereby increasing the efficacy of LLLT. Performing LLLT on clarified tissue requires less power and lower wavelengths from the light source due to the increased penetration of light through the tissue.

In one aspect, a method of performing photobiomodulation of biological tissue in vivo comprises administering a clarifying agent to perfuse a volume of tissue that is underneath and covered by a surface permeability barrier (e.g., stratum corneum of the skin, or conjunctiva of ocular tissues); and applying a source of light energy to the clarified tissue that is covered by a surface permeability barrier.

In another aspect, a system for performing photobiomodulation of biological tissue in vivo comprises an applicator which administers a clarifying agent to a first layer of tissue through a second layer of tissue covering the first layer of tissue with a surface permeability barrier; and a light source which applies light energy to the clarified first layer of tissue.

The use of clarifying agents to change the optical properties of tissue may enhance the efficacy profile of photomodulation procedures. Enhancing the optical transparency of tissue may allow a more pronounced photobiomodulation effect, since a higher dose of the stimulating light irradiation can reach deeper into the tissue, and wavelengths that are believed to produce a higher stimulatory effect, namely lower wavelength light, can penetrate deeper and reach a larger volume of tissue, during the photobiomodulation process. Additionally, most of the key chromophores in the tissue that are stimulated through a photobiomodulation process are significantly more responsive to lower wavelength light. The combination of tissue clearing and photobiomodulation allows a higher dose of light to reach the target chromophores, and also allows lower wavelength light to be used in photobiomodulation, potentially eliciting a much stronger photostimulatory effect. As such, there is a need for enhancing optical transparency of biological tissues during low level light therapy in order to improve the effects of photobiomodulation.

In one aspect, this system and method for photobiomodulation of biological processes uses invasive or non-invasive chemical modification of biological tissues to enhance optical transmission of low-level light energy in sub-epidermal tissue. Other aspects will become more apparent from the specification, drawings, and by reference to the appended claims.

### BRIEF DESCRIPTION OF THE DRAWINGS

FIG. 1 illustrates the optical transmission characteristics of diatrizoate meglumine acid.

FIGS. 2(a), 2(b), and 2(c) illustrate the results of measurement of the diffuse transmission characteristics for porcine sclera, before and after submersion in glycerol, after 5, 10, and 15 minutes, respectively.

FIGS. 3(a), 3(b), and 3(c) illustrate the result of measurement of the diffuse transmission characteristics for porcine sclera, before and after submersion in diatrizoate meglumine acid, after 5, 10, and 15 minutes, respectively.

FIG. 4 illustrates the absorbance of human skin, in-vivo, immediately before and approximately 8 minutes after topical administration of glycerol, over a 460 nm to 800 nm spectral range.

FIG. 5 illustrates the absorbance of human skin, in-vivo, immediately before and approximately 8 minutes after topical administration of diatrizoate sodium injection solution (25%), over a 460 nm to 800 nm spectral range.

FIG. 6 illustrates diagrammatically one embodiment of a generalized system for bypassing surface permeability barrier of tissue, administering a topical chemical, and delivery of light.

FIG. 7 illustrates diagrammatically one embodiment of a pattern for removing the stratum corneum.

FIG. 8 is a flowchart of one embodiment of a diagnostic algorithm.

FIG. 9 is a flowchart of one embodiment of a treatment algorithm.

FIG. 10 is a flowchart of one embodiment of a method of performing photobiomodulation.

FIG. 11 illustrates a step of identifying an area of skin to be treated by photobiomodulation.

FIGS. 12A to 12C illustrate alternative method steps for pre-treating a targeted tissue area in order to bypass the surface permeability layer.

FIGS. 13A to 13C illustrate alternative techniques for applying a clarifying agent to a targeted treatment area.

FIG. 14 illustrates one embodiment of an LED light panel for applying low level laser therapy to a targeted treatment area after pre-treatment to enhance optical transmission of light energy.

### DETAILED DESCRIPTION

Embodiments described herein provide for systems and methods of photobiomodulation through the pre-treatment of in vivo biological tissues with a invasive or non-invasive chemical modification that enhances the optical transmission of light energy through the biological tissues, providing deeper penetration of the light energy and allowing for the use of lower power and lower wavelength light sources at high fluences. The embodiments are useful with low-level light (or laser) therapy (LLLT) using light from across the electromagnetic spectrum, and particularly the visible wavelength range, in order to stimulate or inhibit cell growth.

The range of clinical applications could extend across a large number of conditions that are currently treated with light and non-light-based treatments. These include warts, acne, alteration of fibroblasts to alter the shape and texture of scars, psoriasis, reduction or elimination of unwanted hair, or conversely the stimulation of new hair growth to overcome the effects of alopecia. The above approach could also have a profound effect on treatment of wounds, in augmenting or accelerating the healing of wounds such as chronic venous ulcers, burns, diabetic ulcers, or surgical wounds, and even in deep tissue and vessel diseases such as vessel blockages associated with heart attacks and strokes. Intracellular mechanisms, such as stimulating mitochondrial activity, are considered one recipient of light therapy which may provide a variety of stimulatory effects. The biological effects may be more pronounced than previously observed with LLLT as a result of the deeper penetration of light through the semi-transparent biological tissue. Furthermore, it is believed that lower wavelength light that ordinarily is not able to penetrate deep into the tissue, has a more pronounced stimulatory effect during LLLT, and as such, optical clearing of tissue could enhance the effect of LLLT by allowing such lower wavelength light to reach the target of LLLT within the tissue under treatment.

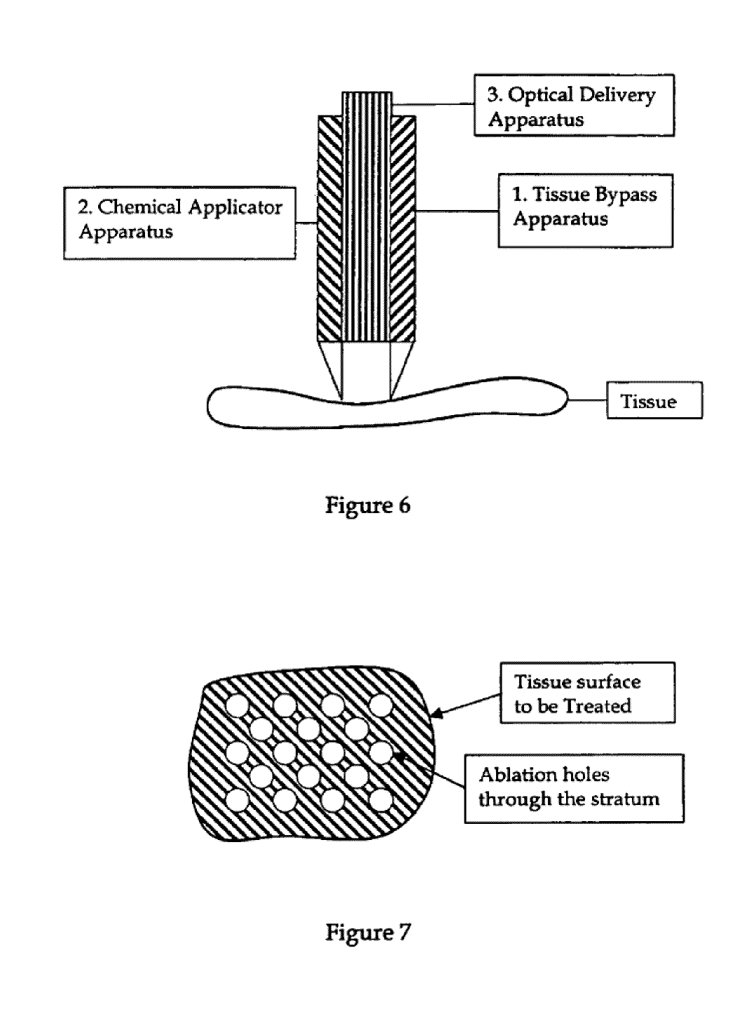

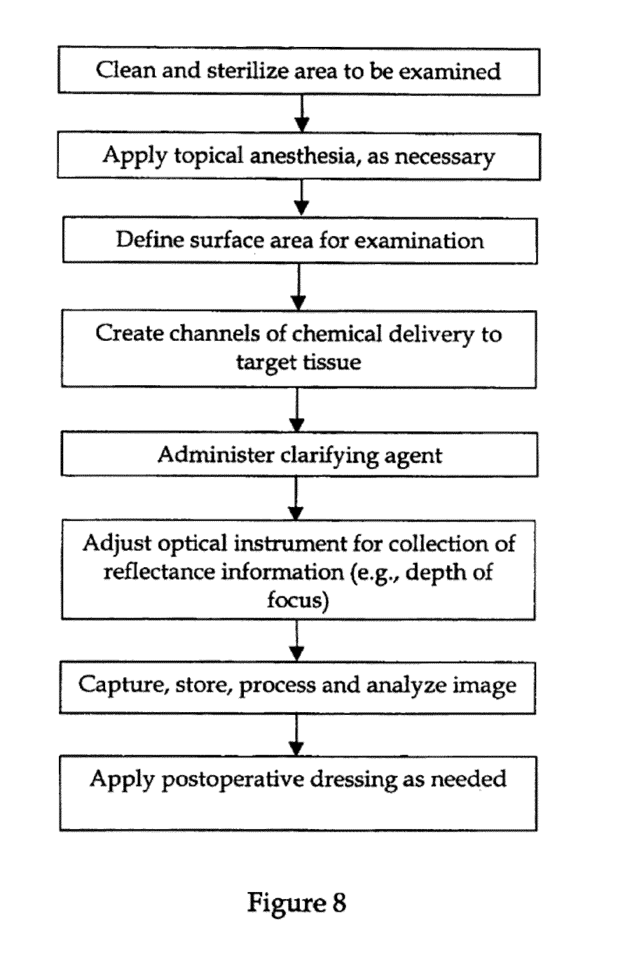

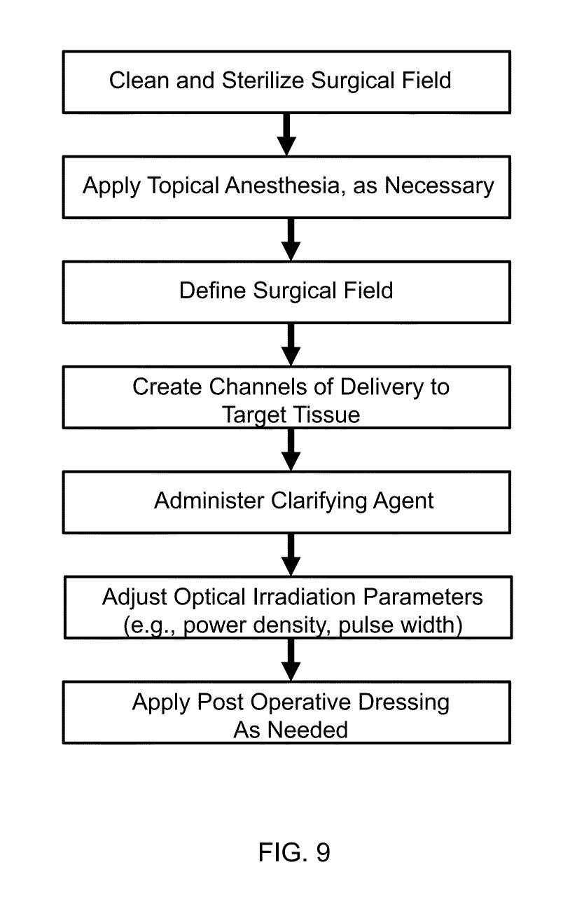

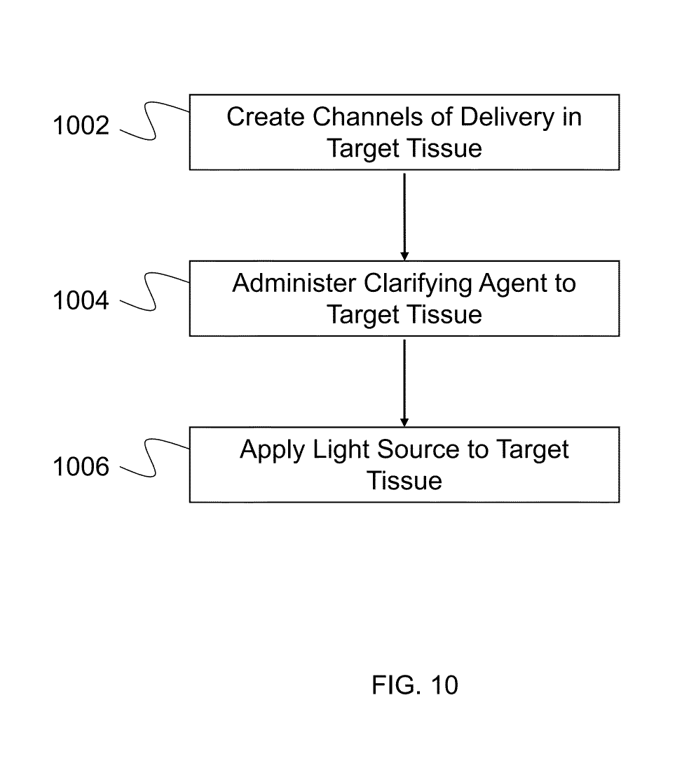

In one embodiment illustrated in FIG. 10, a method of performing photobiomodulation of biological tissue comprises administering a clarifying agent to a first layer of tissue by bypassing a second layer of tissue which covers the first layer with a surface permeability barrier (step 1004). In one optional embodiment, channels of delivery are first created (step 1002) to bypass the surface permeability barrier before the clarifying agent is administered. Specific methods and devices for bypassing the surface permeability barrier and applying a clarifying agent are described in detail further below with reference to FIGS. 6, 7, and 12A to 13C. Once the clarifying agent has entered the tissue underlying the surface permeability barrier, the optical properties of the tissue have been altered and the clarified tissue is now more susceptible to penetration of light energy. A light source operating at low power is then applied to the tissue underlying the surface permeability barrier (step 1006), where it easily penetrates into and through the surface permeability barrier of tissue. As will be described further below, the light source may be one or more of a laser or a plurality of light emitting diodes (LEDs), with the LEDs arranged into an array. The array of LEDs may be arranged to provide exposure over a large surface area or a particular anatomical shape (such as a human face or hand), or it may provide for different LEDs in the array to emit light at multiple wavelengths, in different patterns (such as pulsed or continuous), or for different periods of time. The light source could be a panel of light emitting diodes that could be constructed in an ergonomic conforming form factor to fit the specific anatomy that is to be treated, such as a face mask to be worn for each treatment session for facial treatment, not to exceed 1 hour per treatment session. In this case, the illuminating face mask could be contoured for ergonomics and esthetics to accommodate human use factors. A laser light source may be used for treatment of a much smaller surface area or for therapies that require deeper penetration of light into the tissue. The use of lasers or LEDs as the light source are often in the context of LLLT, which is considered the application of light energy to living tissue for non-thermal or non-ablative uses, such as stimulating or inhibiting cellular growth and other intra-cellular mechanisms. Additional aspects of the use of light sources and LLLT are described in further detail below.

### Improving Optical Transmission

The embodiments described herein are able to change the scattering properties of biological tissues underlying the surface permeability barrier of tissue covering the said biological tissue by changing the refractive index of the interstitial fluid within the stroma and the entire volume of the covered biological tissue.

Optical transmission through biological tissue is one of the major challenges of all optical diagnostic and therapeutic modalities which are intended to access structures underlying the tissue surface. In diagnostics (e.g., imaging tissue structures with microscopes) the object is to obtain a clear image of embedded structures, or signature optical information (e.g., spectroscopic information) from analytes in the blood stream or within the composition of biological tissues. Such images or optical information are distorted due to the attenuation of light, which is transmitted through, or reflected from, the tissue specimens.

In therapeutics (e.g., laser treatment of pigmented and vascular lesions) the goal is to selectively cause thermal necrosis in the target structures, without compromising the viability of surrounding tissues. The highly scattering medium of biological tissues, however, serves to diffuse the incident light, and causes thermal damage to tissues surrounding the target structures, impacting the selectivity of the procedure in destroying the target structures. It is therefore important to adopt a strategy which could minimize the optical attenuation of the overlying and surrounding tissues to treatment target structures, in order to minimize collateral tissue damage, and maximize the therapeutic effects at the target tissue.

When light is irradiated on biological tissues, there are several distinct mechanisms by which light is attenuated. The first attenuation stems from the mismatch of the index of refraction at the tissue interface. This index mismatch results in a reflection from the tissue surface, known as specular reflection. The light that enters into the tissue is also highly scattered and a large portion of such light is ultimately back scattered, after diffusing into the upper layers of the tissue. Scattering within the tissue is a function of the heterogeneity of the refractive indices of the constituents of the tissue, and therefore any effort at reducing this heterogeneity could result in less scattering and therefore a higher level of optical transmission.

The present invention involves the application of a clarifying chemical agent to biological tissues, which are covered by a surface permeability barrier of tissue, in-vivo, ex-vivo, or in-vitro, for the purpose of augmenting the optical transmission through these tissues. While the basis of this effect has not been established definitively, in a number of experiments described below, it has been shown that optical transmission through tissues can be substantially enhanced, on a transient basis, using the topical administration of chemicals such as diatrizoate meglumine acid (commercially known as Hypaque®), glycerol, or glucose (hereinafter referred to as “clarifying agents”). It is possible that the tissue transport phenomena replace a portion of the tissue's water content with the above chemical agents, and the optical properties of these chemicals alter the bulk optical properties of the tissue such that the optical transmission through the tissue is increased. In time, the same transport phenomena replace these agents with water, restoring the tissue's original optical properties.

In the case of these clarifying agents, it is possible that the higher refractive index of these fluids (more than that of water, and closer to the refractive index of collagen and other constituents of tissue), leads to a reduction in the heterogeneity of refractive indices of the constituents of tissue, and therefore reduces the overall scattering of light as it is transmitted through the tissue. This method can be termed “interstitial refractive index matching”, or IRIM.

Glycerol is a trihydric alcohol, a naturally occurring component of body fat. It is absorbed from the gastrointestinal tract rapidly, but at a variable rate. As such, topical administration of glycerol is not expected to cause any adverse effects, and should be a completely safe approach for altering the optical properties of tissues, prior to light irradiation.

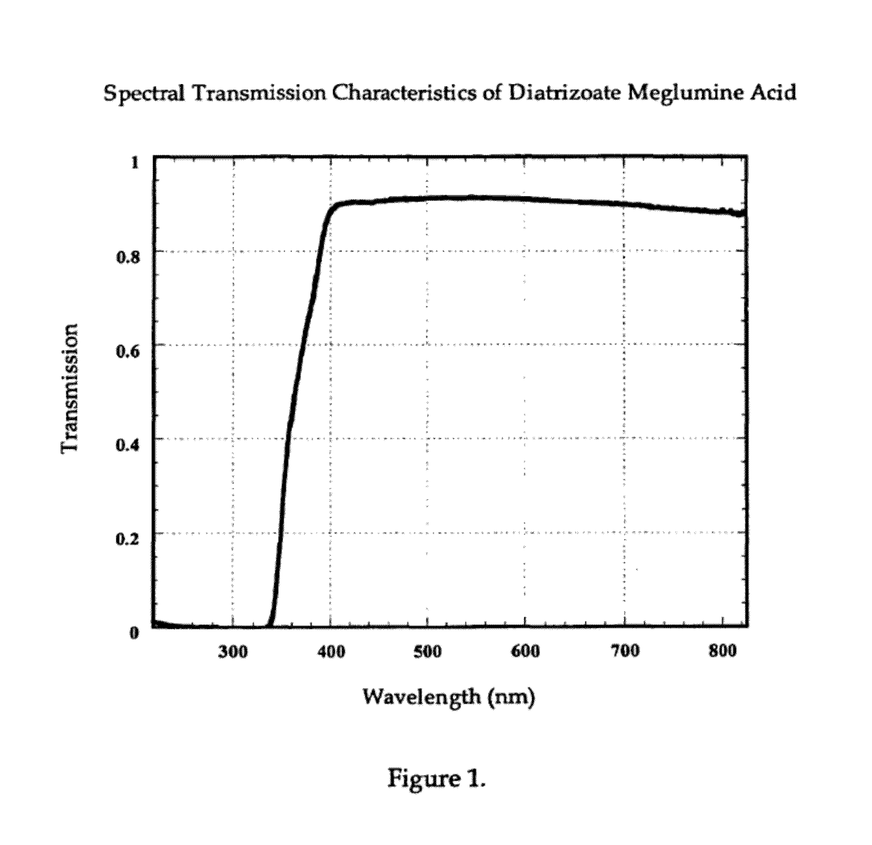

Diatrizoate meglumine acid, which is commercially known as Hypaque®, is a radiographic injection solution. In the case of Hypaque®, the increased transmission through the tissue may be due to IRIM, or optical transmission characteristics which are superior to that of water, or a combination of both effects. In an experiment, a cuvette was filled with diatrizoate meglumine acid and its optical transmission characteristics were evaluated using a Varian CARY 5E™ UV-Vis-NIR spectrophotometer. The transmission characteristics were similar to that of a “high-pass filter”, and the chemical exhibited very low transmission for wavelengths below approximately 400 nm, and very high transmission for wavelengths above 400 nm (see FIG. 1). Experiments demonstrating the effects of IRIM are described below.

### RELEVANT EXPERIMENTS

A. In-Vitro Animal Experiments

Porcine eyes were enucleated immediately post-mortem, and were transported to the laboratory in a wet gauze pad, held at 4° C. during transport in a well-insulated cooler. Each eye was inflated with saline. Limbal conjunctiva and Tenons capsule were dissected and excised using a pair of blunt Wescott scissors. A No. 64 Beaver blade was then used to outline 20 mm×20 mm sections of the sclera from the limbus to the equator of the globe. Incisions were deepened to the supra-choroidal space. Sections of sclera were then lifted off the intact choroid and ciliary body. Scleral thickness was measured and was determined to be 1.65 mm on average.

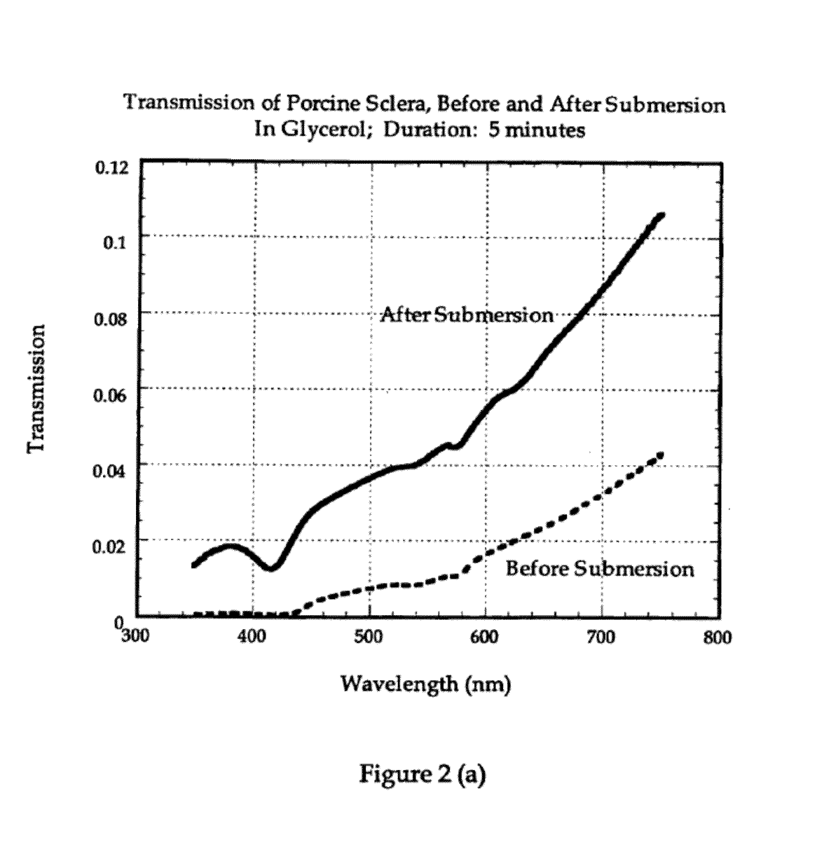

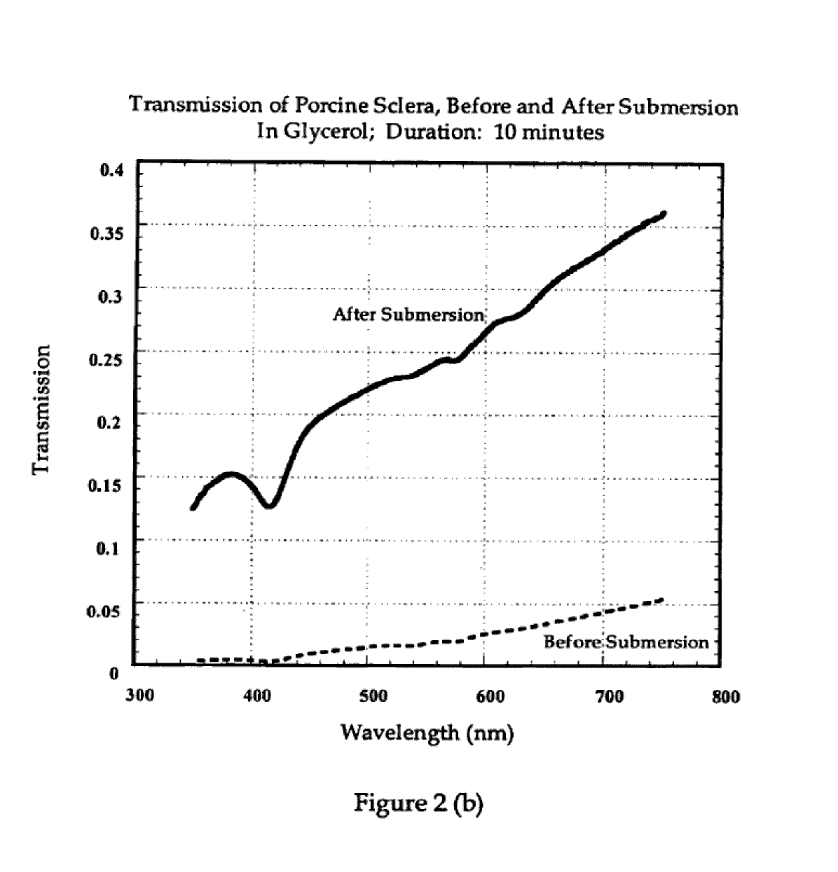

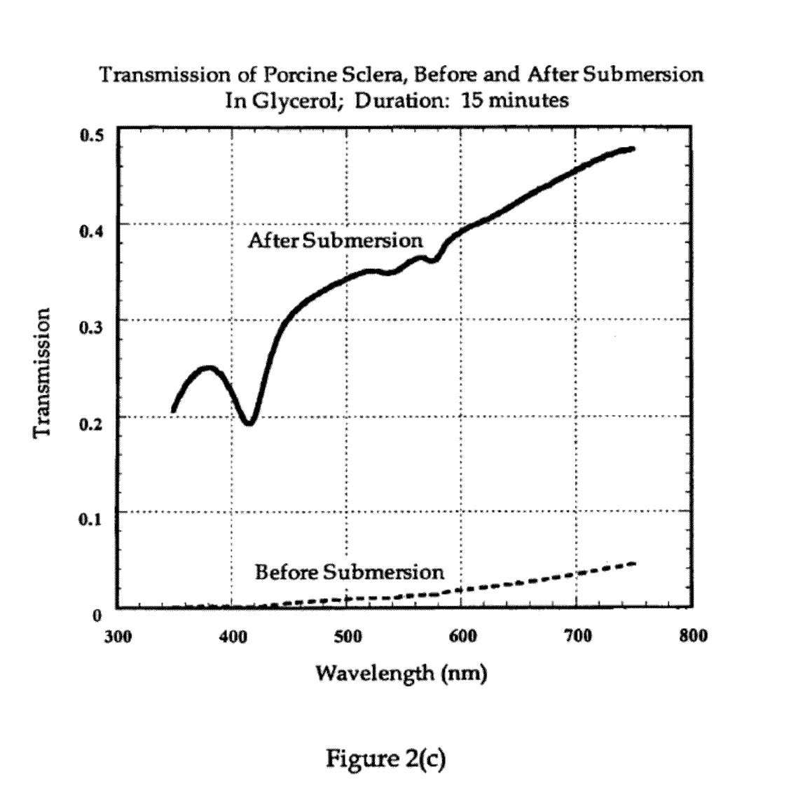

Three prepared sections of sclera were each then placed between quartz slides, and then between two flat aluminum plates. The tissue sample assembly was then fixed against the input port of a diffuse reflectance accessory (integrating sphere) of a Varian Cary 5E™ UV-Vis-NIR spectrophotometer, and diffuse transmission was measured for wavelengths ranging from 350 nm to 750 nm. The three tissue samples were then submerged in separate containers of glycerol, the first being submerged for 5 minutes, the second for 10 minutes, and the third for 15 minutes. Each sample was then again placed in the tissue assembly and was fixed against the input port of the diffuse reflectance accessory of the spectrophotometer, and the diffuse transmission was measured again over the same wavelength range as above. These measurements were also carried out, using three other scleral samples, and diatrizoate meglumine acid as the IRIM agent. The results for these measurements are shown in the FIGS. 2 (a-c), and 3 (a-c).

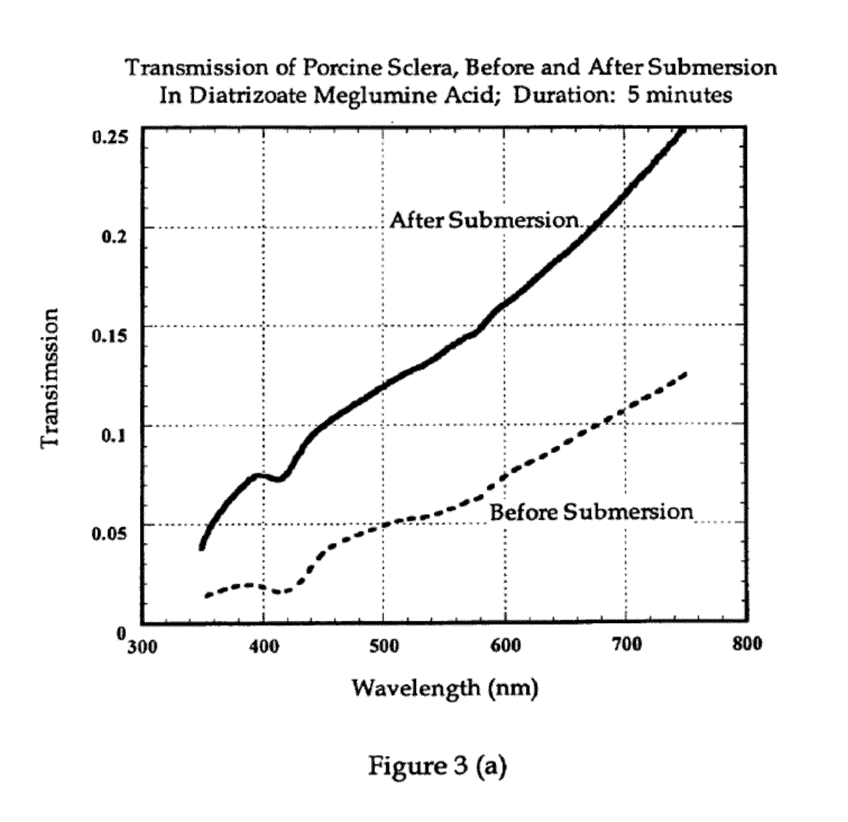

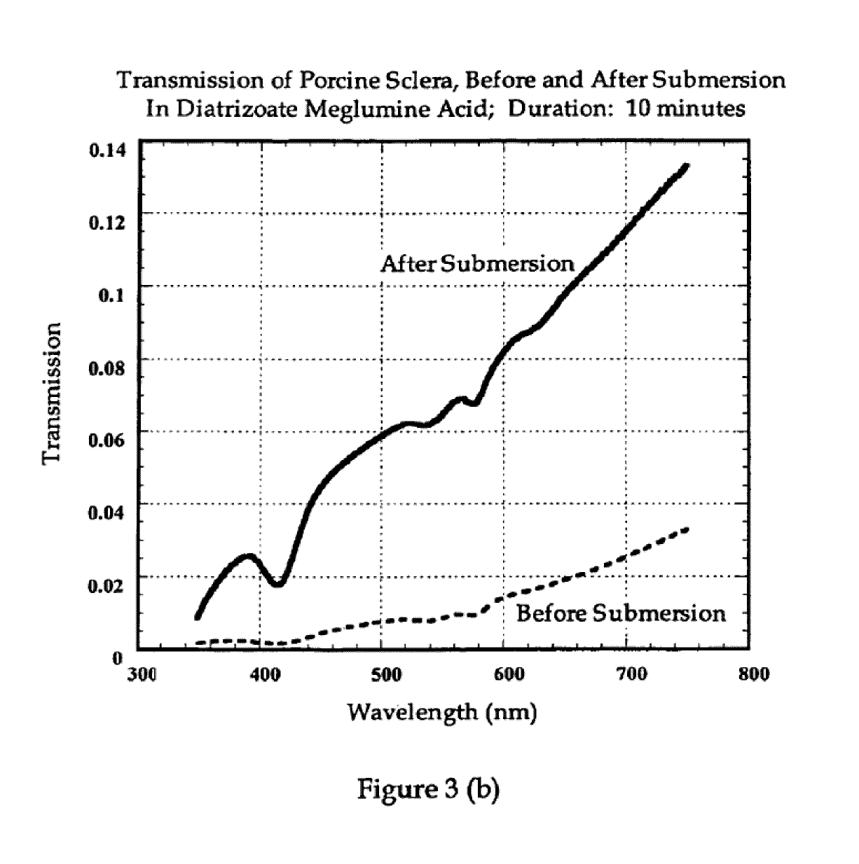

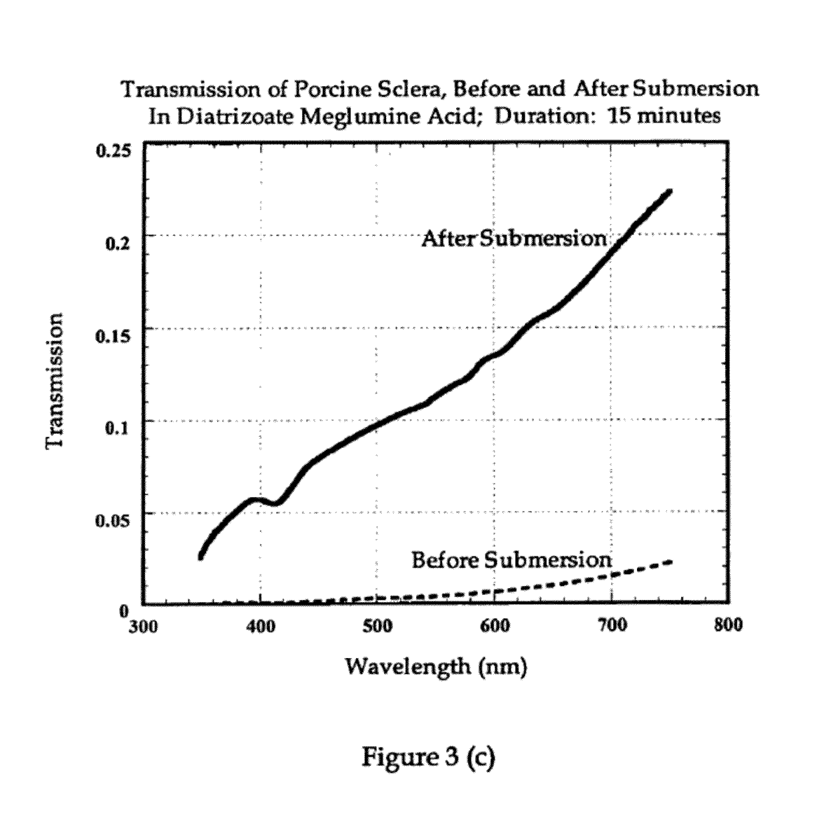

From the results shown in FIG. 2, it can be seen that the diffuse transmission through the sclera was increased by up to 240%, 660%, and 1090%, for samples which were submerged in glycerol for 5, 10, and 15 minutes respectively, with the most pronounced increase occurring at 750 nm wavelength (the longest wavelength for which measurements were carried out). Similarly, the results shown in FIG. 3 illustrate that the diffuse transmission through the sclera was increased by up to 200%, 395%, and 975%, for samples which were submerged in diatrizoate meglumine acid for 5, 10, and 15 minutes respectively, with the largest increase occurring at 750 nm wavelength (the longest wavelength for which measurements were carried out). It is noteworthy that the samples became so transparent (grossly) that when placed against print, letters of the alphabet were clearly discernable through the tissue.

B. In-Vivo Animal Experiments

In order to assess the longitudinal effects of topical administration of glycerol or diatrizoate meglumine acid on the sclera, in-vivo experiments were carried out on two rabbits, and the eyes were examined for signs of inflammation once a day for approximately 1 week.

Each rabbit was put under anesthesia using an intra-muscular injection of Rompin® and Ketamine®. The conjunctiva serves as the permeability barrier for the sclera, and therefore, the conjunctiva was surgically separated from the sclera and the distal surface of the sclera was exposed. Topical drops of glycerol were administered on one eye, and of diatrizoate meglumine acid in the contra-lateral eye, for both rabbits. In the case of glycerol, the sclera turned clear, almost instantaneously (in less than 5 seconds), whereas in the case of diatrizoate meglumine acid, the sclera turned clear in approximately 1 minute. The conjunctiva was stretched over the sclera, again, and stay sutures were used to re-attach the conjunctiva to the limbal region. Ocumycin® was administered topically to both eyes which had undergone surgery, to prevent infection. After approximately 5-10 minutes, the sclera in both eyes became opaque, again. Follow up examination on days 1, 3, and 5 showed no signs of inflammation on either eye.

C. In-Vivo Human Experiments

In order to investigate the applicability of the above method to the human model, and to skin tissue, additional experiments were carried out, using topical glycerol (99.9%, Mallinckrodt, Inc.) and diatrizoate sodium injection fluid, USP, 25% (Hypaque® Sodium, 25%, by Nycomed, Inc., Princeton, N.J.).

Two skin surfaces on the forearm were shaved and cleaned prior to measurements. A tape-strip method was used to remove the stratum corneum. Droplets of glycerol were then topically administered on one site, and droplets of the diatrizoate sodium injection fluid were administered on the second site, which was separated from the first site by 5 cm (a sufficient distance to ensure that the topical drug administered on one site does not interfere with the drug administered on the other site, through diffusion). The topical drugs administered formed an approximate circle of 1 cm in diameter. The drugs were left to diffuse into the tissue for approximately 8 minutes.

Surface reflectance measurements were made immediately after tape stripping of the skin (prior to topical administration of the drugs), and subsequently, approximately 8 minutes after topical administration of the drugs. A CSI Portable Diffuse Reflectance Spectrometer, developed by Canfield Scientific Instruments, Inc. (Fairfield, N.J.), was used for these surface measurements. This device is similar in standard absorption spectrometers, with the exception that the light source is a tungsten halogen lamp, and the sample chamber is replaced with a quartz fiber optic assembly. One leg of the bifurcated fiber optic bundle is coupled to the lamp and the other leg is coupled to the spectrometer. The joined end of the fiber bundle (approximately 3 mm in diameter) was placed in contact with the skin surface from which measurements were made. The measurements were made across a 330 nm to 840 nm spectral range, with a 0.5 nm resolution. The integration time for the measurements was set at 50 kHz.

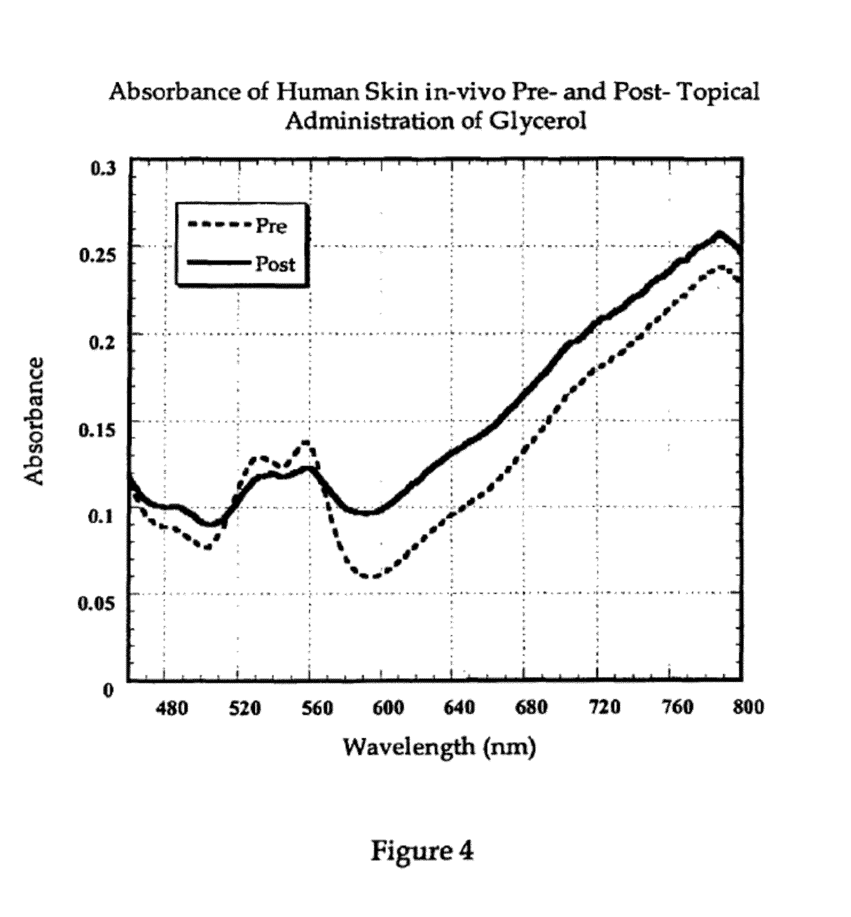

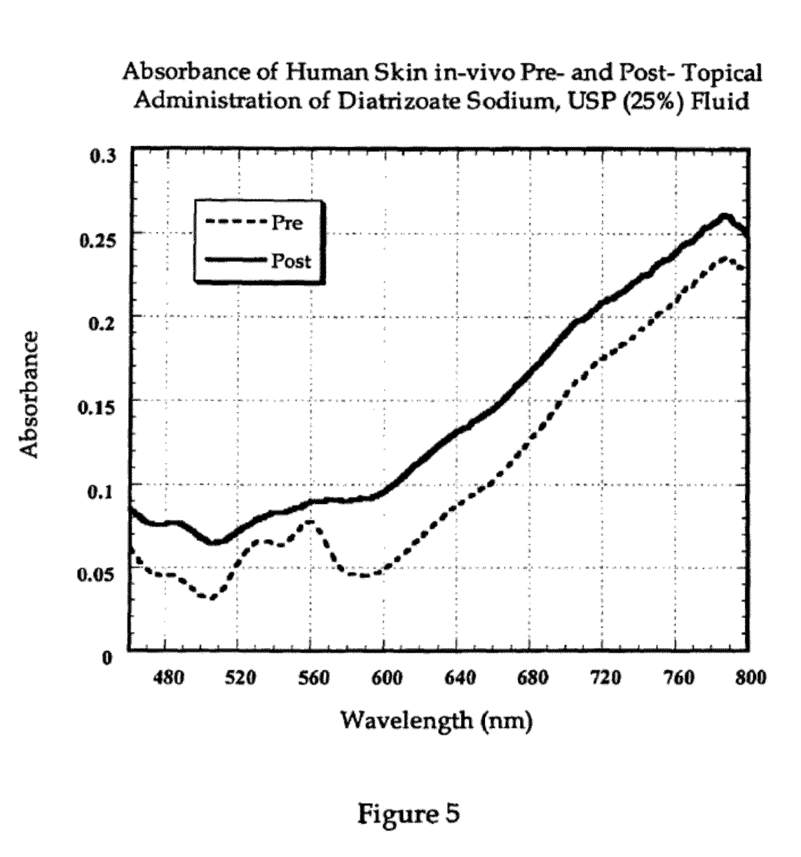

FIGS. 4 and 5 illustrate the results from the above measurements. The data displayed in these graphs have been limited to a range of 480 nm to 800 nm to remove the portions of the spectra which were fraught with noise. The tape stripping of the skin causes a mild irritation of the skin, leading to a transient erythema. This erythema was noticed in both sites immediately after tape stripping, and subsided by the time the measurement after topical administration of the drug was made. As such, the spectra measured prior to topical administration of the drugs clearly demonstrate the higher concentration of blood immediately below the surface, as evidenced by the strong oxyhemoglobin peaks at approximately 530 nm and 560 nm.

FIGS. 4 and 5 demonstrate that the topical administration of glycerol and diatrizoate sodium injection solution, respectively, lead to an increase in absorbance of tissue, in-vivo, of up to 60.5% and 107% respectively. This increase in tissue-absorbance is believed to be caused by IRIM, leading to a reduction of the scattering of the superficial layers of the skin, thereby allowing a larger percentage of light to reach (and get absorbed by) the native chromophores of the skin (melanin and blood), thereby increasing the measured absorbance of the tissue.

It is important to note that further studies are necessary to optimize the above experiments. For instance, the tape-stripping method for removing the stratum corneum is generally unreliable in producing complete removal of this layer. Furthermore, the optimal diffusion time through human skin, for the above drugs, has not yet been determined. As such, the above experiments are only intended as a proof of principle for the use of IRIM in increasing transparency in human tissue, in-vivo. The results, while compelling, are not intended to demonstrate the full potential of this powerful technique in altering the optical properties of human tissue.

### Apparatus Concept

The apparatus comprises the following components: a) apparatus for bypassing the surface permeability barrier of tissue, such as the stratum corneum for the skin, or the conjunctiva for the eye; b) apparatus for topically or interstitially applying a chemical agent; and c) apparatus for delivery or collection of light for diagnostic or therapeutic purposes. Since each of these components consists of devices which are individually known to those skilled in the art, they are shown diagrammatically in FIG. 6. Alternative embodiments may be a combination of all three components, or different combinations of the above in twos.

In order for the topical chemical agent (e.g., glycerol) to affect the tissue stroma (i.e., below the surface layer), it is necessary for this substance to permeate through, or bypass, the surface permeability barrier of tissue. The stratum corneum is a sheet of essentially dead cells which migrate to the surface of the skin. It is well known that dry stratum corneum is relatively impermeable to water soluble substances, and it serves to maintain the hydration of the skin, by providing a barrier for evaporation of the water content of the skin, and also by serving as a barrier for fluids exterior to the body to diffuse into the skin.

Therefore, in order to allow a topically administered chemical agent to be transported into the skin, a strategy needs to be adopted to bypass the stratum corneum. Likewise, the conjunctiva of the eye needs to be bypassed, or surgically removed, for access to the sclera; the same is true for the epithelium of mucosal tissues. Hereinafter, this surface tissue layer will be referred to as the “surface permeability barrier of tissue”, or SPBT, and the underlying tissue layer, as “covered biological tissue” or CBT.

In order to bypass the SPBT, and reach the CBT, a driving force can be applied to move molecules across the SPBT; this driving force can be electrical (e.g., iontophoresis, electroporation) or it may be physical, or chemical force, such as that provided by a temperature gradient, or a concentration gradient of a clarifying agent, or of a carrier agent (carrying clarifying agent) for increasing the permeability of the surface permeability barrier of tissue; alternatively, the driving force may be due to acoustic or optical pressures, as described by Weaver, et al. (Weaver, Powell, & Langer, 1991).

In the case of the stratum corneum, one configuration for component 1 of FIG. 6 can be an electric pulse generator for inducing electroporation of the stratum corneum. This system, for instance, can be similar to the apparatus described by Prausnitz, et al.(Prausnitz et al., 1997).

Alternatively, component 1 may consist of a mechanical device with adhesive tape on the distal end, which may be brought in contact with the skin for tape stripping the stratum corneum. With each adhesion and detachment of the tape from the skin surface, a layer of the stratum corneum can be removed. The component may include means of advancing the tape with each application, so that a fresh tape surface can be used in each application.

More generally, component 1 may consist of a device which physically breaches the surface permeability barrier of tissue by abrasion, to expose the underlying tissue (CBT) which has a greater permeability. In the case of skin, this method is commonly known as dermabrasion.

Alternatively, component 1 may be an ablative solid state laser, such as any one of the following lasers: Er:YAG, Nd:YAG, Ho:YAG, Tm:YAG, Er:YSGG, Er:Glass, or an ablative semiconductor diode laser, such as a high-powered GaAs laser, or an ablative excimer laser, such as an ArFl or a XeCl laser. These lasers can be used to ablate the stratum corneum in its entirety with each pulse, over the surface area covered by the laser spot size. The short ablation depth of such lasers in human tissue (for instance, in the case of Er:YAG, an ablation depth on the order of 5-10 μm) allows for a rapid removal of the stratum corneum with each laser pulse.

Alternatively, component 1 may be an ultrasonic generator, causing poration of the stratum corneum, for instance as described by Kost (Kost et al., 1998). This approach is sometimes referred to as sonophoresis, or phonophoresis.

In yet an alternative configuration, component 1 may be a radiofrequency generator, selectively ablating a finite volume of the stratum corneum with each application, in a similar manner, for instance, as described by Manolis et al. (Manolis, Wang, & Estes, 1994) for ablation of arrhythmogenic cardiac tissues.

Alternatively, component 1 may be a microfabricated microneedle array, long enough to cross the SPBT, but not long enough to reach the nerve endings of the tissue, as that conceived, for instance, by the needle array developed by Henry et al. (Henry, McAllister, Allen, Prausnitz et al., 1998). The clarifying agent can be topically administered onto the SPBT and the microneedle array can then be inserted through the same surface permeability barrier of tissue. The insertion of the needle array will produce an array of apertures through the SPBT, which will then cause an increase in the permeation of the clarifying agent to the covered tissue (CBT).

Alternatively, electrical arcing may be used to ablate the SPBT. This may be done by an electrical generator that delivers electrical arcs at its delivery probe tip. Since stripping a large surface area of the stratum corneum, for instance, may be detrimental for the viability of the skin, the openings may be formed in an array of channels, or apertures, as shown in FIG. 7.

Finally, component 1 may be a dispenser for a chemical enhancer or carrier agent for topical transdermal drug delivery. In the case of the skin, for instance, it has long since been recognized that the permeability of tissue can be increased above its natural state by using penetrating solvents, which when combined with a drug and applied to the skin, greatly increase transdermal drug delivery. An example of one such chemical enhancer is dimethyl sulfoxide (DMSO). Other examples include different alcohols such as ethanol.

In the case of the conjunctiva, it is possible to surgically separate the conjunctiva from the sclera, prior to administration of the topical chemical. Component 1 can consist of a device to create a surgical flap of the conjunctiva, which can subsequently be sutured back onto the intact ocular tissues. Likewise, for the epithelium of mucosa, it is possible to use the same device to create openings in the underlying tissue.

For both the conjunctiva and the mucosa, all of the porative approaches (e.g., electroporation, ultrasonic poration, RF poration, microneedle array, chemical enhancement of trans-membrane delivery, or iontophoresis) may be used as component 1.

In yet another alternative configuration, the chemical may be injected interstitially, using an apparatus similar to a syringe with a hypodermic needle, in order to bypass the surface tissue layer. This approach is more invasive, but the apparatus may be simpler. Component 2 in FIG. 6, is an applicator for administering the chemical topically. One possible configuration is a syringe, which dispenses the desired chemical over the tissue.

Finally, Component 3 of the overall system is the optical delivery or collection apparatus, which may be a fiber optic probe (single or multi-fiber probe), or an articulated arm with specialized optics, depending on the optical delivery system, or optical imaging systems for image acquisition, such as a microscope.

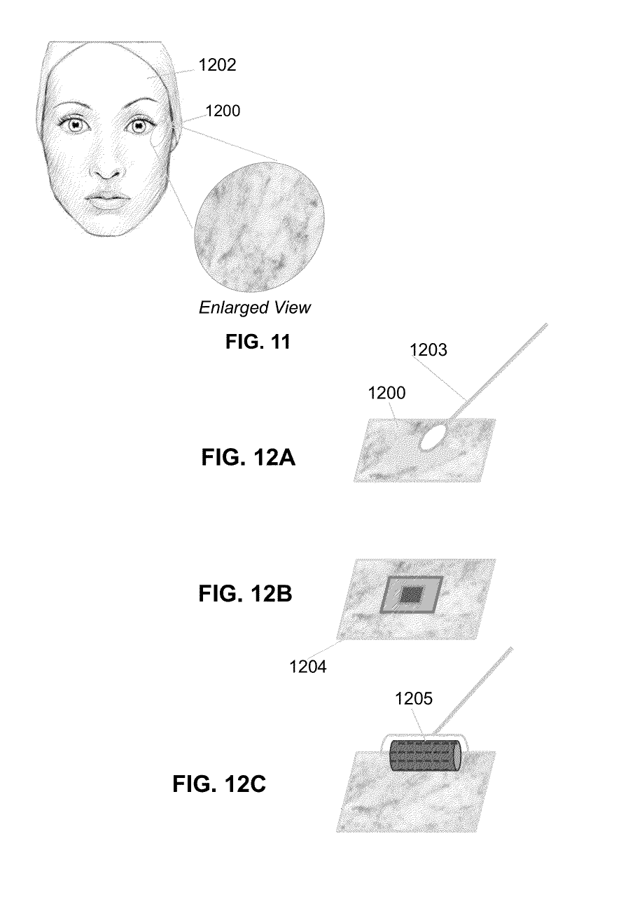

FIGS. 11 to 12C illustrate some alternative embodiments or aspects of a method of creating channels of delivery in targeted tissue in more detail, with FIG. 11 illustrating a first step of identifying, sterilizing and cleaning an area to be treated and FIGS. 12A to 12C illustrating alternative methods for pre-treating targeted tissue in order to bypass the surface permeability barrier. In FIG. 11, an area 1200 to be treated is identified (in this case an area 1200 of face 1202), and the area is cleaned and sterilized as needed. The area may be any part of the body, and the face area 1200 of FIG. 11 is just one example of a possible treatment area.

In the next step, the identified or targeted area is pre-treated before administrating a clarifying agent. FIGS. 12A, 12B and 12C illustrate alternative pre-treatment options. In FIG. 12A, the targeted area 1200 is pre-treated by rubbing with excipients via applicator 1203 to dissolve or abrade the surface permeability barrier. In FIG. 12B, area 1200 is pre-treated by applying a patch 1204 loaded with a selected penetration enhancer to permeabilize the surface permeability barrier, which may be ionic, zwither ionic, or neutral. Some examples of suitable penetration enhancers are sodium lauryl sulfate, sodium octyl sulfate, cetyl trimethyl ammonium bromide, dodecyl pyridinium chloride, octyl trimethyl ammonium bromide, hexadecyl trimethyl ammoniopropane sulfonate, oleyl betaine, cocamidopropyl hydroxysultaine, cocamidopropyl betaine, polyoxyethylene sorbitan monolaurate, sorbitan monolaurate, polyethyleneglycol dodecyl ether, Triton X-100, linoleic acid, linolenic acid, tetracaine, isopropyl myristate, sodium oleate, methyl laurate, N-decyl-2-pyrrolidone, dodecyl amine, nicotine sulfate, menthol, methyl pyrolidone, cineole, limonene, and ethanol, as discussed in more detail below.

In FIG. 12B, the area 1200 is pre-treated by applying a micro-needle array roller 1205 or other means of mechanically creating channels into the surface permeability barrier, forming openings or channels as illustrated in FIG. 7 in the skin surface.

After pre-treatment to create channels of delivery, clarifying agent is administered to the target tissue. The clarifying agent may be any of the agents dsicussed above, and may be administered in a number of different ways, including topically or via physical, chemical or thermal force, as further described in detail below. One example of administering the clarifying agent is illustrated in FIG. 6. In this example, the tissue bypass apparatus includes a chemical applicator for applying the clarifying agent after piercing the surface permeability barrier.

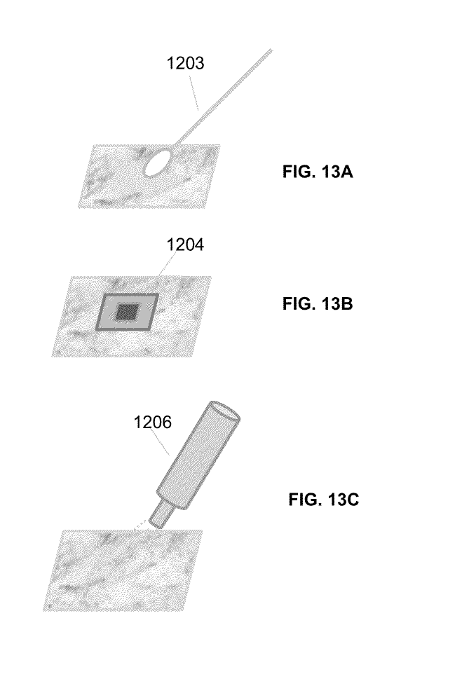

FIGS. 13A to 13C illustrate other alternative methods for applying the clarifying agent in step 1004 of FIG. 10. In the first option of FIG. 13A, a suitable applicator 1203 is used to apply the clarifying agent to area 1200. The clarifying agent may include a penetration enhancing formulation to increase penetration, such as any of the penetration enhancers listed above. As noted above, the step of bypassing the surface permeability barrier in one embodiment may involve abrasion of the surface permeability barrier, such as abrasion of the stratum corneum of the skin, in order to enhance the topical administration of the clarifying agent. In the option of FIG. 13B, the clarifying agent is applied using an occlusive or non-occlusive patch 1204, loaded with the clarifying agent with or without penetration enhancers. An apparatus used for such a treatment could involve a patch that uses a physical, chemical, or thermal means to topically administer a clarifying agent to the targeted regions. In the alternative of FIG. 13C, clarifying agent is applied using a pressurized injection device 1206 or other means of delivering the clarifying agent by physical, chemical, or thermal force.

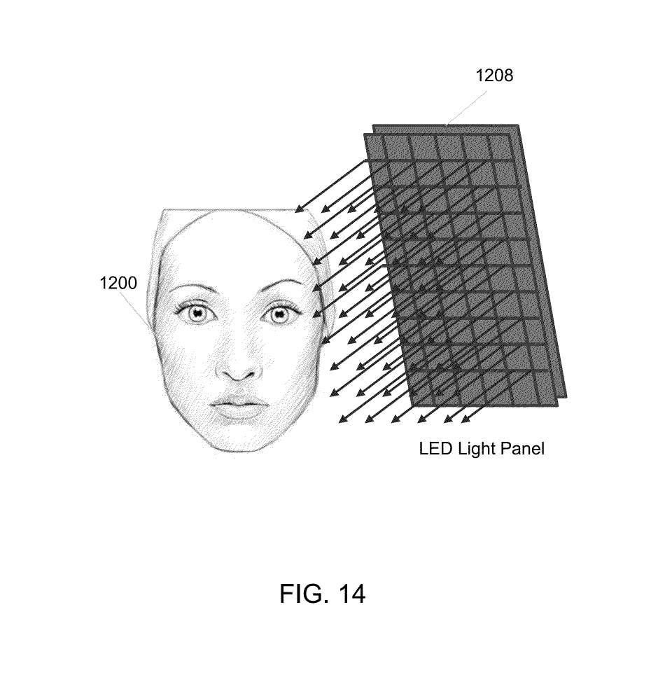

The light source used in step 1006 for application of light to the treated target tissue area 1200 may be one or more separate types of light sources arranged in unique configurations and arrays and varying in power and wavelength in order to achieve a particular biological effect through photobiomodulation. FIG. 14 illustrates one embodiment in which an LED light panel 1208 is used to administer light to a facial area for photobiomodulation. LED arrays of different shapes and sizes may be used depending on the configuration of the target tissue area.

### Applications

Since virtually all diagnostic and treatment procedures in the field of biomedical optics involve the probing of light into biological media, the invention described here has broad applications across a wide range of optical procedures. The method described here essentially serves to augment optical penetration of biological tissues.

A. Therapeutics

The present method is applicable to a wide array of therapeutic means involving embedded objects in the tissue. In the field of ophthalmology, it encompasses all transscleral procedures, including transscleral cyclophotocoagulation, and transscleral retinopexy.

In the field of dermatology, applications include (but are not limited to) skin rejuvenation, optical (or laser) targeting of all skin appendages, including the hair follicle (for stimulating hair growth), pigmented and vascular lesions, sebaceous glands (for acne treatment), subcutaneous fat (for optical liposuction), and eccrine glands (for permanent treatment of body odor).

The present method could also be used to enhance any and all treatments using infra red laser energy to treat a broad range of disease and health conditions, such as treatment of ischemic stroke using transcranial laser therapy.

The systems and methods described herein also improve the efficacy of diagnostic and therapeutic procedures, when energy sources from other segments of the electromagnetic spectrum are used (e.g., radiofrequency, and microwaves). Since IRIM also affects the overall elastic properties of tissues, acoustic signals traveling through tissue could also be affected, leading to a deeper penetration for ultrasonic waves for diagnostic and therapeutic purposes.

The range of clinical applications could extend across a large number of conditions that are currently treated with light and non-light based treatments. These include warts, acne, alteration of fibroblasts to alter the shape and texture of scars, psoriasis, reduction or elimination of unwanted hair, or conversely the stimulation of new hair growth to overcome the effects of alopecia. The above approach could also have a profound effect on treatment of wounds, in augmenting or accelerating the healing of wounds such as chronic venous ulcers, burns, diabetic ulcers, or surgical wounds, and even in deep tissue and vessel diseases such as vessel blockages associated with heart attacks and strokes. Intracellular mechanisms, such as stimulating mitochondrial activity, are considered one recipient of light therapy which may provide a variety of stimulatory effects. The biological effects may be more pronounced than previously observed with low-level light (or laser) therapy (LLLT) as a result of the deeper penetration of light through the semi-transparent biological tissue.

C. Further Contemplated Uses and Compositions

In further contemplated embodiments of the inventive subject matter, only partial clarification is performed on a variety of biological tissues, and especially contemplated tissues include those that are accessible from the outside of a mammal, and particularly a human. Thus, suitable target tissues include skin, sclera, mucous membranes, lingual tissue, and the tympanic membrane. Partial clarification is particularly advantageous to reduce thermal damage to a tissue component. It should be especially noted that, in the embodiment pertaining to laser irradiation of a target object within tissue, while tissue clarification is employed, the tissue component that comprises the target object of laser irradiation remains substantially unclarified. Using such partial clarification, it is contemplated that the thermal damage to the irradiated tissue mayl be substantially reduced, if not even entirely avoided.

In one embodiment of a method of irradiating a target object in skin, a clarification agent in a topical formulation is provided. In another step it is ascertained that the target object is located in a sub-papillary layer of the skin, wherein the target most typically comprises an endogenous or exogenous chromophore. In yet another step, the clarifying agent is topically applied to at least one layer of epidermis and/or the dermal papillary layer under a protocol effective to achieve clarification of the layer of epidermis and/or the papillary layer. Notably, contemplated protocols will provide substantially no clarification of the sub-papillary layer. In a still further step of contemplated methods, the skin is then irradiated with laser irradiation having visible light emission at a wavelength of less than 700 nm and at an energy effective to at least partially destroy the target object, wherein the step of irradiating is performed under a protocol effective to avoid thermal damage in the layer of epidermis and the papillary layer.

For better reference, the following description of the various layers of skin is provided following a view from the outside to the inside of a body: The stratum corneum, with a characteristic layer of dead cells, is the surface permeability barrier that cover the epidermis, the outmost layer of skin and generally thinner than the dermis. Depending on the location, the thickness will vary. In certain locations (e.g., lips, palms), the stratum lucidum is found below the stratum corneum, while the malpighian layer (typically including the stratum granulosum and stratum spinosum) is generally present throughout the body and located below the stratum corneum and stratum lucidum. The stratum germinativum provides the germinal cells necessary for the regeneration of the layers of the epidermis and is located directly below the malpighian layer.

Following the stratum germinativum are the dermal layers that make up the dermis that is generally comprised of vascularized, dense, irregular connective tissue with primarily type I collagen and elastin fibers. In most locations, blood vessels perfuse part of the dermis, which is divided into two anatomically distinct regions, the papillary dermis and the sub-papillary reticular dermis: The papillary dermis is composed of loose connective tissue (typically comprising thin bundles of collagen mixed with elastin, fibrocytes and stromal matrix), capillaries and Meissner's corpuscles that project into the dermal papillae. The reticular layer is below the papillary layer and contains dense irregular connective tissue (typically comprising thicker bundles of collagen and elastin, fewer fibrocytes and stromal matrix), blood vessels (e.g., vascular plexus that supplies dermal papillae as well as the eccrine and folliculosebaceous glands), lymph vessels, adipocytes, hair follicles, and nerves.

Below the dermis is the hypodermis, which comprises a layer of loose connective tissue immediately deep to the dermis of the skin. The hypodermis typically includes loosely arranged elastic fibres and fibrous bands anchoring the skin to deep fascia. The hypodermis further includes various fatty deposits, blood vessels on route to dermis, lymphatic vessels on route from dermis, hair follicle roots, the glandular part of some sudiferous glands, and neural structures (e.g., free endings, and/or Panicinian corpuscles).

In one aspect, the target object comprises an endogenous or exogenous chromophore. For example, where the chromophore is melanin, the target object is a hair papilla or hair follicle (wherein additional pigments or dyes may be supplied to the follicle using methods well known in the art). In another example, the pigment may be a metal containing tattoo pigment. However, it should be appreciated that non-pigmented target objects are also contemplated herein and that such objects especially include collagen and elastin in the reticular layer. Therefore, the target object is most typically located in a sub-papillary layer of skin, and even more typically in a reticular layer of the dermis and/or the hypodermis. The location of the target object can typically be determined from sources well known in the art, or be determined using skin biopsy and light microscopy following well established procedures. For example, it is well known that the hair follicles and/or hair papillae are located in the sub-papillary layer (here: the reticular layer of the dermis), while the location of a tattoo pigment may be ascertained by biopsy and light-microscopy. Further contemplated target objects include pigmented lesions other than tattoos, wherein the pigment may be of natural origin (most typically from within the body in which the pigmented lesion is found). For example, alternative pigmented lesions include age spots, hyperpigmented areas, melasmas, as well as blood vessels, and vascular lesions.

With respect to the clarification agent, all of the above discussed agents are deemed suitable for use herein. However, especially preferred clarification agents include those that are pharmaceutically acceptable and metabolized and/or excreted within a relatively short period (e.g., 50% metabolized/excreted within 24 hours) of time. Among other suitable agents, particularly preferred clarification agents include polyols (e.g., glycerol), diatrizoate meglumine acid, and glucose (dextrose). Further preferred agents and aspects of such agents are disclosed in U.S. Pat. No. 6,275,726 to Chan, which is incorporated by reference herein. Suitable concentrations of clarification agents will be in the range of between about 5-95 wt %, more typically between 10-85 wt %, and most typically between about 30-75 wt % of the topical formulation.

Contemplated clarification compositions and formulations can be prepared using various protocols, and a particular composition typically determines (at least in part) a particular protocol. There are numerous methods and protocols known in the art, and exemplary protocols and formulations are described in “Topical Drug Bioavailability, Bioequivalence, and Penetration” by Vinod P. Shah, Howard I. Maibach (Editor), Plenum Pub Corp; ISBN: 0306443678, or in “Percutaneous Penetration Enhancers” by Eric W. Smith (Editor), Howard I. Maibach (Editor), CRC Press; ISBN: 0849326052, or in “Pharmaceutical Skin Penetration Enhancement” by Kenneth A. Walters, Jonathan Hadgraft (Editor), Marcel Dekker; ISBN: 0824790170, or in “Drug Permeation Enhancement: Theory and Applications” by D.S. Hseih, Ed. (Dekker, New York, 1994), all of which are incorporated by reference herein.

Consequently, contemplated compositions and formulations are typically preparations for topical application, and particularly include preparations in form of a cream, gel, lotion, ointment, salve, or a paste. Alternatively, contemplated compositions and formulations may also include preparations in liquid form (e.g., a syrup, tincture, spray, drops, etc.), all of which may or may not be applied with a patch, for example a patch 1204 as illustrated in FIG. 13B and described in more detail above. Most preferably, such formulations will include a penetration enhancer, which may be ionic, zwitter ionic, neutral, etc. Therefore, contemplated penetration enhancers include sodium lauryl sulfate, sodium octyl sulfate, cetyl trimethyl ammonium bromide, dodecyl pyridinium chloride, octyl trimethyl ammonium bromide, hexadecyl trimethyl ammoniopropane sulfonate, oleyl betaine, cocamidopropyl hydroxysultaine, cocamidopropyl betaine, polyoxyethylene sorbitan monolaurate, sorbitan monolaurate, polyethyleneglycol dodecyl ether, Triton X-100, linoleic acid, linolenic acid, tetracaine, isopropyl myristate, sodium oleate, methyl laurate, N-decyl-2-pyrrolidone, dodecyl amine, nicotine sulfate, menthol, methyl pyrolidone, cineole, limonene, and ethanol.

Depending on the particular type of topical formulation, removal of at least one epidermal layer (e.g., stratum corneum) and/or dermal layer (typically papillary layer) may be removed. There are numerous manners of such removal known in the art, and all of those are deemed suitable for use herein. Among other methods, epidermal layers can be removed using tape stripping, dermabrasion, laser resurfacing, chemical peels, etc. Alternatively, contemplated clarification agents may also be injected or otherwise transported to mechanically or optically disrupted epidermal/dermal layers. However, in one aspect, the clarification agent is topically applied without removal the stratum corneum and/or stratum lucidum.

Where desirable, contemplated formulations (with or without penetration enhancer) may also be delivered to the site of clarification using methods and devices that increase the rate of delivery to the tissue that is to be clarified. Among other contemplated devices and methods, the delivery of the clarification agent may be assisted by heat (e.g., chemically or electrically generated), electrical current (e.g., electrophoresis, iontophoresis, electroporation, etc.), pressure (e.g., using ultrasound or low-frequency [<20 kHz] vibration), and occlusion (e.g., under film or bandage).

Application quantities, area, and duration may vary considerably in different embodiments, depending on the delivery method and technique for assisting delivery. In one embodiment, the application of the topical formulation precedes laser irradiation, and the formulation is applied for a time period equal or less than 2 hours, more typically less than 60 minutes, and most typically less than 30 minutes prior to light irradiation. Furthermore, and again depending on the type of application, it is generally contemplated that the topical formulation is applied to an area of at least 50 cm², more typically at least 100 cm², and most typically at least 300 cm². Duration of application may vary between several minutes and several hours, and more typically between 10 minutes and 120 minutes, and most typically between 10 minutes and 60 minutes. The amount of clarification agent applied is selected to be sufficient to clarify at least one layer of the target tissue, and optionally in the skin the papillary layer of the dermis, while providing substantially no clarification of the sub-papillary layer. The term “substantially no clarification of the sub-papillary layer” as used herein means that the difference in light reflection, refraction, and/or scattering between treated and untreated skin and with respect to the sub-papillary layer is less than 20% of the maximum achievable value, and more typically less than 10% of the maximum achievable value as can be measured by methods well known in the art. Viewed from another perspective, the target structure remains in the reticular or other tissue layer in a substantially unclarified, and more typically entirely unclarified environment. In some embodiments no clarification is provided in the reticular layer, while in other aspects some clarification (equal or less than 10% of maximum clarification), while in still other aspects minor clarification (equal or less than 20% of maximum clarification) is provided in the reticular layer. Thus, appropriate amounts of topical formulation applied vary depending on the amount of clarification desired in the respective tissue layer. In some embodiments suitable quantities of clarification agent are in the range of between 5 microgram and 100 milligram, and may be between 50 microgram and 10 milligram in one specific example.

### Low-Level Light Therapy (LLLT)

Although there is no universally accepted definition or parameters for low-level light therapy (LLLT), it is generally considered to be the use of light at levels which do not create any thermal or evaporative (ablative) effect on the biological living tissue, and instead provides a stimulatory effect through an intracellular biochemical reaction, otherwise known as photobiomodulation. Therefore, the specific power, wavelengths and energy of LLLT light sources may vary as long as the physiological effect of the light source remains primarily biochemical. However, one of skill in the art will appreciate that the methods and specifications of LLLT light sources described herein are not limited to use only with LLLT, and may provide therapeutic, medical or other physiological benefits known or unknown.

In one embodiment, the light source may be a low level laser light or at least one light emitting diode (LED). In one embodiment, the LEDs may be arranged in an array to provide a cumulative effect of all of the LEDs, for example as illustrated in FIG. 14, and may be arranged to provide varied wavelengths simultaneously, to provide various patterns. In one embodiment, LED arrays which are arranged to fit a particular anatomical feature, such as a human face, hand, etc. are provided. The method of light exposure could be pulsed or through a continuous wave light source.

The wavelength of light to be applied may range from visible to infra-red radiation. Although higher wavelengths of red light and infrared light have accepted LLLT uses, the effects of the clarifying agents on the sub-epidermal layers may provide uses for blue green light. As mentioned above, optical properties of biological tissues suggest that most key components of biological tissue serve as chromophores that are more responsive to lower wavelength light. However, this lower wavelength light has previously been unable to penetrate deep enough into tissue to produce any potential beneficial effects. The use of the clarifying agents described above therefore expands the wavelengths of light which can be used for LLLT. Additionally, red light (generally wavelengths of 620-750 nm), which normally is difficult to penetrate into biological tissues, may be even more effective as a result of the application of the clarifying agent.

In one embodiment, the energy level (or light energy density) of the light source (measured as energy=power×time) is no greater than approximately 4 J/cm² (Joules per centimeter squared).

In one embodiment, the skin or other tissue is irradiated with a laser light (or other light source, preferably with monochromatic or narrow band [equal or less than 100 nm bandwidth] filtered light) in the visible and/or near infrared range having a per square centimeter intensity of less than 100 J/cm², more typically less than 75 J/cm², even more typically less than 60 J/cm², and most typically between 10 W and 45 W. Suitable light sources may provide a radiation energy level of between 5-60 J/cm². There are numerous medical light sources, and especially light sources for the treatment of skin associated conditions known in the art (e.g., continuous, pulsed, etc.), and all of such light sources are contemplated herein. In some embodiments light sources may include those with an emission wavelength of between 430 nm to 700 nm, including green-blue light sources and red light sources. It should be recognized that the particular choice of light source depends at least in part on the particular purpose.

It should be particularly noted that by clarification of tissue layers above the reticular layer, inadvertent damage is reduced in such tissue layers. Moreover, as such tissue layers cause less loss of light intensity due to scattering, reflection, and/or refraction, it should be noted that lasers and LED arrays can be used at reduced power output and/or exposure.

Thus, specific embodiments and applications of the systems and methods to enhance optical transparency of biological tissues for photobiomodulation have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Furthermore, where a definition or use of a term in a reference, which is incorporated by reference herein is inconsistent or contrary to the definition of that term provided herein, the definition of that term provided herein applies and the definition of that term in the reference does not apply.

### REFERENCES

Lee, S. Y., Park, K-H, Choi, J-W, et al., A prospective, randomized, placebo-controlled, double-blinded, and split-face clinical study on LED phototherapy for skin rejuvenation: Clinical, profilometric, histologic, ultrastructural, and biochemical evaluations and comparison of three different treatments, J. Photochem. Photobiol. B: Biology, Vol. 88, Issue 1, July 2007, pp. 51-67.

McDaniel, David H., Method and Apparatus for the Photomodulation of Living Cells, U.S. Pat. No. 6,663,659, Issued Dec. 16, 2003.

McCarthy, J. J., Fairing, J. D., & Buchholz, J. C. (Inventors). (1989 Feb. 7). I. Tracor Northern (Assignee). Confocal tandem scanning reflected light microscope. (U.S. Pat. No. 4,802,748).

Lucas, F. F. (1930). The architecture of living cells—recent advances in methods of biological research—optical sectioning with the ultra-violet microscope. N.A.S.

Chan, K. F., Nemati, B., Rylander, I. H. G., & Welch, A. J. (1996). Chemically enhanced scleral transmission: a new approach for transscleral cyclophotogcoagulation. Proceedings of the Fourteenth Annual Houston Conference on Biomedical Engineering Research Houston.

Vogel, A., Dlugos, C., Nuffer, R., Birngruber, R. et al. (1991). Optical Properties of human sclera and their significance for transscleral laser applications. Laser Surg Med, 11(4), 331-340.

Cantor, L. B., Nichols, D. A., Katz, L. J., Moster, M. R., Poryzees, E., Shields, J. A., & Spaeth, G. L. (1989). Neodymium-YAG transscleral cyclophotocoagulation. The role of pigmentation. Investigative Ophthalmology and Visual Science, 30(8), 1834-1837.

Flood, T. P. et al. (1989). Hyperosmotic Agents. Duane's Biomedical Foundation of Ophthalmology (Vol. 3p. 5). Philadelphia: J. B. Lippencott Company.

Weaver, J. C., Powell, K. T., & Langer, R. S. Jr. (Inventors). (1991 May 28). Massachusetts Institute of Technology (Assignee). Control of transport of molecules across tissue using electroporation. (U.S. Pat. No. 5,019,034).

Prausnitz, M. R. et al. (1997). Reversible skin permeabilization for transdermal delivery of macromolecules. Crit Rev Ther Drug Carrier Syst, 14(4), 455-483.

Kost, J. et al. (1998). Phonophoresis. B. Berner, Dinh Steven M. et al. (Editors), Electronically controlled drug delivery (pp. 215-228). Boca Raton, Fla.: CRC Press, Inc.

Manolis, A. S., Wang, P. J., & Estes, N. A. 3. (1994). Radiofrequency catheter ablation for cardiac tachyarrhythmias. Annals of Internal Medicine, 121(6), 452-461.

Henry, S., McAllister, D. V., Allen, M. D., Prausnitz, M. R. et al. (1998). Microfabricated microneedles: a novel approach to transdermal drug delivery. T. Pharm Sci, 87(8), 922-925.

What is claimed is:

1. A method of performing photobiomodulation of biological tissue in vivo, comprising:
administering a clarifying agent to a first layer of biological tissue through a surface permeability barrier layer of tissue covering the said biological tissue; and
applying a source of light energy to the clarified covered biological tissue.

2. The method of claim 1, wherein the clarification agent comprises at least one of glycerol, diatrizoate meglumine acid, and glucose.

3. The method of claim 2, wherein the clarifying agent is administered through at least one of a physical, chemical or thermal force.

4. The method of claim 2, wherein the clarifying agent is administered topically.

5. The method of claim 4, wherein the step of topically administering the clarifying agent is performed without removal of the surface permeability barrier.

6. The method of claim 4, wherein the clarifying agent further includes a penetration enhancer.

7. The method of claim 1, wherein the source of light energy is one or more light emitting diodes (LEDs).

8. The method of claim 7, wherein a plurality of LEDs are arranged into an array.

9. The method of claim 8, wherein the plurality of LEDs operate at a power density of approximately 5 J/cm² to approximately 100 J/cm².

10. The method of claim 1, wherein the source of light energy is a laser, a flash lamp light source, or a light emitting diode.

11. The method of claim 1, wherein the light source operates at a power of approximately 5 mW (milliwatts) to approximately 10 W.

12. The method of claim 1, wherein the light energy has a wavelength of approximately 330 nanometers (nm) to approximately 840 nm.

13. The method of claim 1, wherein the light energy has a wavelength of approximately 600 nm-950 nm.

14. The method of claim 1, wherein the light source is a low level light source having a light energy density no greater approximately 4 J/cm².

15. A system for performing photobiomodulation of biological tissue in vivo comprising:
an applicator which administers a clarifying agent to a first layer of tissue through a second layer of tissue covering the first layer of tissue with a surface permeability barrier; and
a light source which applies light energy to the clarified first layer of tissue.

16. The system of claim 15, wherein the clarification agent comprises at least one of glycerol, diatrizoate meglumine acid, and glucose.

17. The system of claim 15, wherein the applicator administers the clarifying agent through at least one of a physical, chemical or thermal force.

18. The system of claim 15, wherein the applicator administers the clarifying agent topically.

19. The system of claim 18, wherein the applicator administers the clarifying agent without removal of at least one of a stratum corneum layer of the second epidermal layer and a stratum lucidum layer of the second epidermal layer.

20. The system of claim 18, wherein the clarifying agent further includes a penetration enhancer.

21. The system of claim 15, wherein the light source is one or more light emitting diodes (LEDs).

22. The system of claim 21, wherein the light source is a plurality of LEDs arranged into an array.

23. The system of claim 15, wherein the light source is a laser.

24. The system of claim 15, wherein the light energy has a wavelength of approximately 330 nanometers (nm) to approximately 840 nm.

25. The system of claim 15, wherein the light energy has a wavelength of approximately 600 nm-950 nm.

26. The system of claim 15, wherein the light source is a low level light source having a light energy density of less than or equal to approximately 4 J/cm².
